(19) United States
(12) Patent Application Publication
(10) Pub. No.: US 2026/0345258 A1
(43) Pub. Date: Nov. 5, 2026

(54) ARRANGEMENT OF AN ELECTROSURGICAL INSTRUMENT

(71) Applicant: CMR Surgical Limited, Cambridge (GB)

(72) Inventors: Matthew David Knight, Sunnyvale, CA (US); Max Julian L'Huillier Jansa, London (GB)

(73) Assignee: CMR Surgical Limited, Cambridge, OT (GB)

(21) Appl. No.: 19/124,450

(22) PCT Filed: Oct. 30, 2023
(86) PCT No.: PCT/GB2023/052827

§ 371 Date: Apr. 25, 2025

(30) Foreign Application Priority Data
Oct. 31, 2022 (GB) 2216134.3

(51) Int. Cl. A61B 34/30; A61B 34/00

(52) U.S. Cl. CPC A61B 34/30; A61B 34/71; A61B 2034/305

(57) ABSTRACT

A robotic electrosurgical instrument includes a shaft, an end effector including an electrical component, wherein the end effector includes opposing first and second end effector elements, a first joint permitting the end effector to rotate relative to the shaft about a first axis, a first driving element configured to drive the end effector about a second joint, the passage of the first driving element through the instrument defining a first path that extends at least partially around the first joint, and an electrical cable configured to provide electrical current to the electrical component, the passage of the electrical cable through the instrument defining a second path that is offset from the first path along the first axis and that extends parallel to the first path around the first joint.

### BACKGROUND OF THE INVENTION

This disclosure relates to a robotic electrosurgical instrument.

Electrosurgery is continually becoming more frequently used within the field of surgical robotics due to the enhanced functionality and reduced blood loss advantages achieved through the use of electrosurgical instruments. Electrosurgery is a term used to define surgical operations that are performed using instruments that are powered by a high frequency alternating electrical current that is used to heat surgical tissue.

Electrosurgical instruments that are attached to surgical robots typically comprise an end effector that is connected to a shaft via one or more articulations, or joints. The end effector may be expected to adopt a number of different rotational configurations during its use in a surgical procedure. The end effector of an instrument is typically supplied with electrical current by one or more electrical cables that are connected to the end effector of the instrument, the cables receiving current from an electrical power source that is comprised within or connected to the surgical robot.

There are various areas of development for robotic electrosurgical instruments. A significant area of development is in the configuration of electrical cables through the electrosurgical instrument so that those cables are able to withstand the different strains and configurations that are effected during robotic surgery.

### SUMMARY OF THE INVENTION

According to a first aspect, there is provided a robotic electrosurgical instrument comprising: a shaft; an end effector comprising an electrical component; a first joint permitting the end effector to rotate relative to the shaft about a first axis; a first driving element configured to drive the end effector about a second joint, the passage of the first driving element through the instrument defining a first path that extends at least partially around the first joint; and an electrical cable configured to provide electrical current to the electrical component, the passage of the electrical cable through the instrument defining a second path that is offset from the first path along the first axis and that extends parallel to the first path around the first joint.

The first and second paths may be at least partially circumferential around the first joint.

The first joint may comprise a first pulley that is rotatable about the first axis, the first pulley comprising a first groove, wherein the first driving element is at least partially housed within the first groove.

The first pulley may further comprise a second groove that is offset from and axially aligned with the first groove along the first axis, and the electrical cable may be at least partially housed within the second groove.

The first pulley may have a circular cross-sectional area in a plane that is perpendicular to the first axis.

Each of the first and second grooves may extend around an outer circumference of the first pulley.

The path of the first groove around the outer circumference of the first pulley may have a radius that is the same as the radius of the path of the second groove around the outer circumference of the first pulley.

The path of the first groove around the outer circumference of the first pulley may have a radius that is smaller than the radius of the path of the second groove around the outer circumference of the first pulley.

The first joint may further comprise a second pulley, the second pulley comprising a third groove that is offset from and axially aligned with the first groove along the first axis, and the electrical cable may be at least partially housed within the third groove.

The second pulley may be rotatable about the first axis independently of the first pulley.

The second pulley may have a circular cross-sectional area in a plane that is perpendicular to the first axis.

The third groove may extend around an outer circumference of the second pulley.

The path of the first groove around the outer circumference of the first pulley may have a radius that is the same as the radius of the path of the third groove around the outer circumference of the second pulley.

The path of the first groove around the outer circumference of the first pulley may have a radius that is smaller than the radius of the path of the third groove around the outer circumference of the second pulley.

The instrument may further comprise a third pulley that is positioned between the first and second joints and is angularly offset from the first and second joints, wherein the second path extends at least partially around the third pulley.

The first driving element may be a cable.

The end effector may comprise opposing first and second end effector elements, the first end effector element being rotatable about the second joint and the second end effector element being independently rotatable relative to the shaft about a third axis by means of a third joint.

The electrical component may be a first electrical component comprised within the first end effector element and the electrical cable may be a first electrical cable being configured to provide electrical current to the first electrical component, and the surgical instrument may further comprise: a second driving element configured to drive the third joint, the length of the second driving element defining a third path that extends at least partially around the first joint; a second electrical component comprised within the second end effector element; and a second electrical cable configured to provide electrical current to the second electrical component; wherein the passage of the second electrical cable through the instrument defines a fourth path that is offset from the third path along the first axis and extends parallel to the third path around the first joint.

The first and second end effector elements may be first and second jaws of the end effector.

The first joint may be driven by a pair of driving elements comprising the first driving element and a first further driving element, wherein the length of the further driving element defines a fifth path that extends at least partially around the first joint.

The instrument may further comprise a resilient barrier inside the shaft extending over a cross-sectional area of the shaft, wherein each of the first and second driving elements is configured to pass through respective first and second channels that extend through the resilient barrier, and the electrical cable may be configured to pass through a third channel that extends through the resilient barrier.

The first, second and third channels may be cylindrical in shape, and a centre point of the third channel may be offset from a line that passes through (a) a centre point of the first channel and (b) a centre point of the second channel, along a distal surface of the resilient barrier.

The first joint may comprise the first pulley and a further pulley that is also rotatable about the first axis, the further pulley comprising a further groove configured to at least partially house the second driving element; and the instrument may further comprise a fourth joint located parallel to the first axis and offset from the first axis along the longitudinal axis of the shaft, wherein the fourth joint comprises a first pulley configured to house the first driving element and no further pulley configured to house the second driving element.

The instrument may be configured to be connected to a surgical robot.

### BRIEF DESCRIPTION ON THE FIGURES

The present invention will now be described by way of example with reference to the accompanying drawings. In the drawings:

FIG. 1 illustrates a surgical robot;

FIG. 2 illustrates a first exemplary robotic surgical instrument for use with a surgical robot;

FIG. 3 further illustrates the exemplary robotic surgical instrument of FIG. 2;

FIG. 4 illustrates a second exemplary robotic surgical instrument for use with a surgical robot;

FIG. 5 further illustrates the exemplary robotic surgical instrument of FIG. 4;

FIG. 6 illustrates a pulley for use in the robotic surgical instrument illustrated in FIGS. 3 and 4;

FIG. 7 illustrates two pulleys for use in the robotic surgical instrument illustrated in FIGS. 3 and 4;

FIG. 8 illustrates the shaft of an exemplary robotic surgical instrument;

FIG. 9 illustrates a first exemplary resilient barrier for a surgical instrument;

FIG. 10 illustrates a second exemplary resilient barrier for a surgical instrument;

FIG. 11 illustrates a third exemplary robotic surgical instrument;

FIG. 12 illustrates a third exemplary resilient barrier for a surgical instrument.

### DETAILED DESCRIPTION

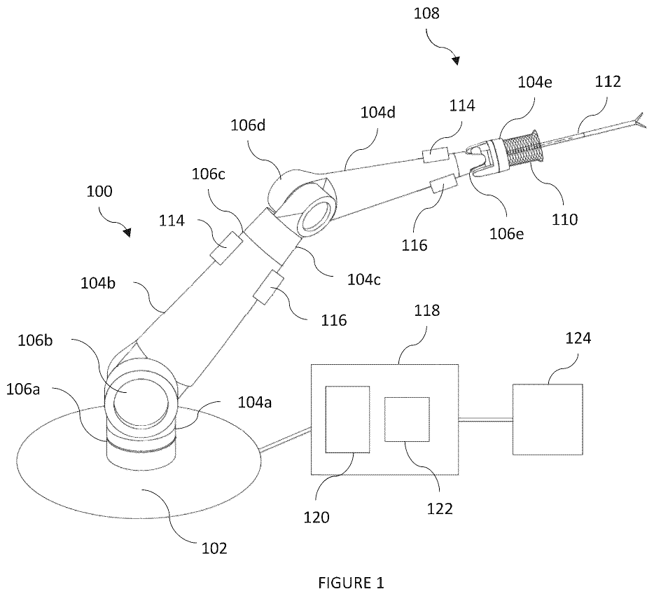

FIG. 1 illustrates a surgical robot having an arm 100 which extends from a base unit 102. The arm comprises a plurality of rigid limbs 104a-e which are coupled by a plurality of joints 106a-e. The joints 106a-e are configured to apply motion to the limbs. The limb that is closest to the base 102 is the most proximal limb 104a and is coupled to the base by a proximal joint 106a. The remaining limbs of the arm are each coupled in series by a joint of the plurality of joints 106b-e. A wrist 108 may comprise four individual revolute joints. The wrist 108 couples one limb (104d) to the most distal limb (104e) of the arm. The most distal limb 104e carries an attachment 110 for a surgical instrument 112. Each joint 106a-e of the arm 100 has one or more drive sources 114 which can be operated to cause rotational motion at the respective joint. Each drive source 114 is connected to its respective joint 106a-e by a drivetrain which transfers power from the drive source to the joint. In one example, the drive sources 114 are motors. The drive sources 114 may alternatively be hydraulic actuators, or any other suitable means. Each joint 106a-e further comprises one or more configuration and/or force sensors 116 which provides sensory information regarding the current configuration and/or force at that joint. In addition to configuration and/or force sensory data, the one or more sensors 116 may additionally provide information regarding sensed temperature, current or pressure (such as hydraulic pressure).

The arm terminates in an attachment for interfacing with the surgical instrument 112. The surgical instrument has a diameter less than 8 mm. The surgical instrument may have a 5 mm diameter. The surgical instrument may have a diameter which is less than 5 mm. The surgical instrument comprises an end effector for performing an operation. The end effector may take any suitable form. For example, the end effector may be smooth jaws, serrated jaws, a gripper, a pair of sheers, a needle for suturing, a camera, a laser, a knife, a stapler, a cauteriser or a suctioner. The surgical instrument further comprises an instrument shaft and an articulation located between the instrument shaft and the end effector. The articulation comprises one or more joints that permit the end effector to move relative to the shaft of the instrument. The joints in the articulation are actuated by driving elements. These driving elements are secured at the other end of the instrument shaft to interface elements of an instrument interface. The driving elements are elongate elements that extend from the joints in the articulation through the shaft to the instrument interface. Each driving element can be flexed transverse to its longitudinal axis in the specified regions. In an example, the driving elements may be cables.

The diameter of the surgical instrument may be the diameter of the profile of the articulation. The diameter of the profile of the articulation may match or be narrower than the diameter of the shaft. The attachment comprises a drive assembly for driving articulation of the instrument. Movable interface elements of the drive assembly interface mechanically engage corresponding movable interface elements of the instrument interface in order to transfer drive from the robot arm to the instrument. Thus, the robot arm transfers drive to the end effector as follows: movement of a drive assembly interface element moves an instrument interface element which moves a driving element which moves a joint of the articulation which moves the end effector.

Controllers for the drive sources 114 and sensors 116 are distributed within the robot arm 100. The controllers are connected via a communication bus to a control unit 118. The control unit 118 comprises a processor 120 and a memory 122. The memory 122 stores, in a non-transient way, software that is executable by the processor 120 to control the operation of the drive sources 114 to cause the arm 100 to operate. In particular, the software can control the processor 120 to cause the drive sources (for example via distributed controllers) to drive in dependence on inputs from the sensors 116 and from a surgeon command interface 124.

The surgical instrument may be an electrosurgical instrument. The term “electrosurgical instrument” within the context of this application is used to refer to an instrument that has an end effector with an electrical component to which electrical current is to be provided for the correct operation of the surgical instrument. For example, an electrosurgical instrument can perform electrosurgery, electrocautery, diathermy or any other form of surgical procedure involving the operation of an electrical component in the end effector, e.g., for heating of surgical tissue using electrical current. The end effector(s) of an electrosurgical instrument may be similar to those of surgical instruments that are not capable of performing electrosurgery. For example, the end effector of an electrosurgical instrument may be smooth jaws, serrated jaws, a pair of shears, a knife, or a cauteriser. As it is suitable for attachment to a surgical robot, the electrosurgical instrument may be referred to as a robotic electrosurgical instrument.

If the end effector comprises one or more electrically conducting components (e.g., metal components such as metal jaws or a metal blade) then these one or more electrically conducting components may be the “electrical component(s)” to which electrical current is to be provided. For example, the end effector of an electrosurgical instrument may comprise an electrical component configured to receive electrical current and to transform that current into an alternative type of energy that is used to generate heat. The alternative type of energy may be radio frequency (RF) energy, microwave energy, ultrasound energy or energy of another suitable wavelength that is able to heat surgical tissue. The electrical component may be an electrode or a type of emitter of energy waves. The type of wave emitted by the electrical component is dependent on the frequency of the electrical current that is provided to the end effector. As another example, the end effector of an electrosurgical instrument may comprise an electrical component configured to receive electrical current and to conduct that current so that it passes into the surgical tissue of the patient, wherein when the electrical current flows through the surgical tissue it generates heat. An end effector may comprise more than one type of electrical component. For example, an end effector may comprise both an emitter of microwaves and an electrode. The different types of electrical components may be used for different purposes. For example, a first type of electrical component may be used to cut surgical tissue and second type of electrical component may be used to seal this tissue. Where the end effector comprises more than one type of electrical component, the electrical cables required to supply electrical current to each electrical component may be of the same type or of different types, depending on the requirements for the respective types of energy emitted by the components.

The instrument is supplied with electrical current by at least one electrical cable that extends along (e.g., through) the shaft. The electrical cable is attached at its first end to the instrument and at its second end to a source of electrical current (e.g., alternating current), that may be part of and located at the base of the surgical robot, for example. In an alternative example, the source of electrical current may be a standalone unit that is separate to the surgical robot. The electrosurgical instrument further comprises an electrical connector 230 that provides an electrical connection between the electrical cable and the electrical component of the end effector(s).

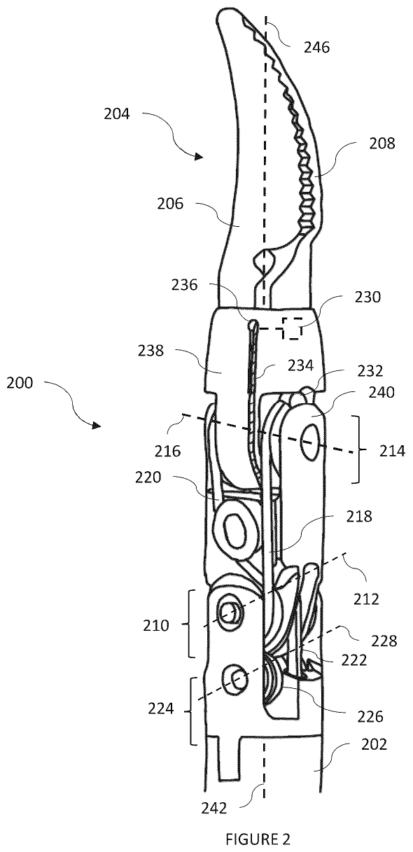

An example of the distal end of a robotic electrosurgical instrument 200 is illustrated in FIG. 2. The robotic electrosurgical instrument 200 is configured to be connected to a surgical robot. The instrument comprises a shaft 202 at its proximal end (i.e., the end closest to the connection to a robot arm) and an end effector 204 at a distal end that opposes the proximal end. The end effector 204 has a pair of end effector elements 206, 208. The end effector 204 is connected to the distal end of the shaft 202 of the instrument by an articulation. The shaft 202 is connected at its proximal end to an interface for attaching to a robot arm. The articulation comprises joints 210, 214, 224 that permit movement of the end effector 204 relative to the shaft 202.

The shaft 202 terminates at its distal end at a first joint 210. The first joint 210 is comprised within the articulation. The first joint 210 permits the end effector 204 to rotate about a first axis 212. The first joint 210 may be referred to as a rotational joint. A rotational joint shall for the purposes of this application be defined as a joint that allows two bodies to rotate relative to each other about a common axis. A rotational joint may comprise a plurality of components. The articulation comprises a supporting body 240. At a first end, the supporting body 240 is connected to the shaft 202 by the first joint 210. At a second end opposing the first end, the supporting body 240 is connected to the end effector 204 by at least a second joint 214. The instrument illustrated in FIG. 2 is in a straight configuration. In this configuration, the end effector 204 is aligned with the shaft 202. That is, in the straight configuration the longitudinal axis 246 of the end effector is coincident with longitudinal axis 242 of the shaft.

The second joint 214 permits the first end effector element 206 of the end effector 204 to rotate about a second axis 216. The second axis 216 may be transverse to the longitudinal axis 242 of the shaft 202. A third joint (not illustrated) permits the second end effector element 208 to rotate about a third axis (not illustrated). The third axis may also be transverse to the longitudinal axis 242 of the shaft. The further third may be parallel to the second axis 216. In the example illustrated in FIG. 2, the second and further axes are the same axis, i.e., they are collinear. However, in alternative examples, the third axis is not the same as the second axis. For example, the third axis may be parallel to but offset from the second axis 216. The offset may be in a direction defined by (e.g., along) the longitudinal axis 242 of the shaft. The offset may be in a direction that is not defined with respect to (e.g., not along) the longitudinal axis 242 of the shaft.

The first end effector element 208 and the second end effector element 206 may be independently rotatable about the second and third axes respectively because of the second and third joints. The end effector elements may be rotated in the same direction or different directions by the second and third joints. The second axis 216 is transverse to the first axis 212. The second joint 214 and third joint permit the end effector elements 206, 208 to rotate relative to the supporting body about the second and third axes 216.

The surgical instrument may further comprise a fourth joint 224. The fourth joint 224 may comprise at least one pulley 226. The fourth joint is located relative to the first joint 210 so as to ensure that the components that drive the first joint are retained in contact with the first joint 210. The pulleys of the fourth joint 224 may rotate about a fourth axis 228. The fourth axis 228 may be parallel to the first axis 212. The fourth axis 228 is offset from the first axis 212 along the longitudinal axis 242 of the shaft.

Each joint of the instrument is driven by at least one driving element. Each joint of the instrument may be driven by a pair of driving elements. Each joint of the instrument may be independently driven. The first joint 210 is driven by at least one driving element 222. The second joint is driven by at least one driving element 218. The third joint is driven by at least one driving element 220. Thus, the first, second and third joints of the instrument are independently driven. The driving elements are elongate elements which extend from the joints in the articulation through the shaft to the instrument interface. Suitably, each driving element can be flexed laterally to its main extent at least in those regions where it engages the internal components of the articulation and instrument interface. In other words, each driving element can be flexed transverse to its longitudinal axis in those specific regions. This flexibility enables the driving elements to wrap around the internal structure of the instrument. The driving elements may be wholly flexible transverse to their longitudinal axes. The driving elements are not flexible along their main extents. The driving elements resist compression and tension forces applied along their length. In other words, the driving elements resist compression and tension forces acting in the direction of their longitudinal axes. Thus, the driving elements are able to transfer drive from the instrument interface to the joints. The driving elements may be cables.

The second joint 214 is driven by a first driving element 218. In other words, the first driving element 218 is configured to drive the second joint 214. The elongate nature of the driving elements is such that the passage of each element through the instrument defines a path that extends at least partially around a relevant joint of the instrument. For example, the passage of the first driving element 218 through the instrument defines a first path that extends at least partially around the second joint 214. The second joint 214 may be driven by a pair of driving elements comprising the first driving element 218 and a further driving element (not illustrated). Similarly, the third joint (not illustrated) may be driven by a second driving element 220. The third joint may be driven by a pair of further driving elements comprising the second driving element 220 and a further driving element (not illustrated). The first joint 210 is driven by a third driving element 222. The third driving element 222 extends at least partially around the first joint 210 so as to drive the first joint. The first joint 210 may be driven by a pair of driving elements comprising the third driving element 222 and a further driving element (not illustrated). The one or more driving elements configured to drive each joint of the instrument may be secured to their corresponding joint. For example, the first driving element(s) 218 may comprise a ball feature, or crimp, 232 that is secured to the second joint 214. This ensures that when the driving elements(s) are driven, the drive is transferred to motion of the joint about the second axis 216. A corresponding ball feature is secured to each of the third joint and first joints to ensure that drive is transferred to motion of those joints about their respective axes.

The end effector elements 206, 208 are illustrated in FIG. 2 as being a pair of opposing serrated jaws. However, the end effector elements may take any alternatively suitable form such as smooth jaws, a pair of shears or a pair of blades of a gripping tool. Whilst the end effector of the instrument illustrated in FIG. 2 comprises two end effector elements, it is appreciated that in alternative examples the instrument may comprise a single end effector element, or more than two end effector elements.

The instrument further comprises at least one electrical cable 234. In some examples, the instrument may comprise a single electrical cable 234. The single electrical cable 234 provides electrical energy to the end effector 204. In other examples, the instrument may comprise two electrical cables. In these examples, each electrical cable may supply electrical energy to a respective electrical component of an end effector comprising two end effector elements. The electrical cable 234 is configured to provide electrical current to the end effector. The electrical cable 234 is connected to the end effector at a first end by an electrical connector 230. The electrical cable 234 may be connected at a second end to the driving elements of the surgical instrument. More specifically, the electrical cable 234 may be connected at its second end to spokes of the instrument. The spokes are rigid tubes that increase the stiffness of the driving elements. The spokes are located within the shaft 202 of the instrument. The spokes are located proximally of the end effector 204 of the instrument.

The electrical connector 230 may plug directly into the end effector 204. The electrical connector 230 may provide a fixed electrical connection between the electrical cable 234 and the end effector 204. That is, when connected to the end effector 204, the end effector 204 may not be able to move independently of the electrical connector. Alternatively, the electrical connector 230 may provide a sliding electrical connection between the electrical cable 234 and the end effector 204. That is, the end effector 204 may be able to move independently of the electrical connector when the connector is connected to the end effector 204.

The end effector comprises at least one electrical component (e.g., the metal jaws of the end effector elements 206 and 208) configured to receive electrical current from the electrical cable 234 and, for example, transform that current into energy that is used to generate heat. Energy from the electrical component is transferred to a patient via the end effector element 206, 208. In one example, the electrical component is an electrode. Where the electrical component is an electrode, the electrode is configured to heat surgical tissue by ohmic heating when electrons pass through the tissue. Alternatively, if the electrical component is an emitter of energy waves, the waves are transferred to a part of the end effector element that is configured to contact the body of a patient. The waves are in turn configured to heat and vaporise the water content of surgical tissue. The waves generated by the emitter may for example be radio frequency (RF) waves, microwaves, infrared waves, infrared waves, ultrasound waves or waves of any other suitable frequency that can be used to heat organic tissue during a surgical procedure. The type of wave emitted may be dependent on the frequency of current that is provided to the end effector. The instrument also comprises an external insulator 238 configured to insulate the electrically charged components of the instrument such as the electrical connector and the electrical component from other parts of the surgical instrument and from the patient. The external insulator 238 may comprise a groove 236 within which the electrical cable sits. The groove 236 may define a path for the electrical cable 234 from the second/third joints to the end effector elements of the end effector 204.

Where the instrument comprises a single electrical component, it is described as a monopolar instrument. For a monopolar instrument, the single electrical component of the electrosurgical instrument may be an electrode that is configured to contact the tissue of a patient and heat the tissue at a first location on the patient. The electrode of the monopolar instrument may be referred to as an “active” electrode. A second electrode may be external to the instrument and connected to a second location on the patient, and to disperse current from the active electrode from the patient. The second electrode may be referred to as a “dispersive” electrode. The monopolar instrument requires a single electrical cable to provide electrical current to its single electrical component. Thus, in turn, a monopolar instrument requires a single electrical connector to provide an electrical connection between its electrical cable and electrical component.

In an alternative example, such as the example illustrated in FIG. 2, the instrument may comprise a pair of electrical components. In this example, the electrosurgical instrument is described as a bipolar instrument. A first electrical component may be positioned on a first side of the end effector, and a second electrical component may be positioned on a second side of the end effector that opposes the first side. Where an end effector comprises two end effector elements, a first electrical component may be (or may be attached to) the first end effector element and a second electrical component may be (or may be attached to) the second end effector element. Where the end effector comprises two end effector elements, alternating energy waves may oscillate between the two end effector elements when the electrical components are charged, heating the intervening tissue by oscillation of intracellular ions.

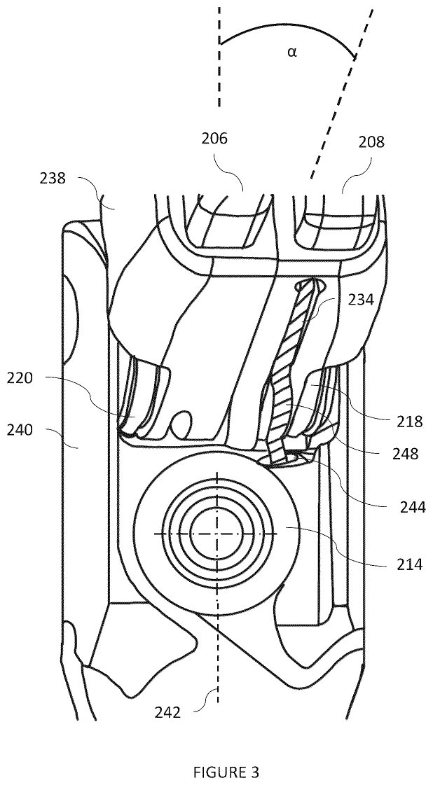

It is mentioned above that the driving elements of the surgical instrument 200 are attached to the end effectors at their distal end by a ball feature, or crimp (e.g., crimp 232). The electrical cables of the instrument are attached to the end effectors at their distal end by an electrical connector (e.g., electrical connector 230). It can be seen from FIG. 2 that the electrical connector 230 is located distally to the crimp 232 along the surgical instrument. Thus, the path length of the electrical cable 234 from the spoke of the driving element (which is the point of attachment of the electrical cable) through the instrument is different to the path length of the first driving element 218 through the instrument from the spoke. More specifically, the path length of the electrical cable from the spoke to its end effector is longer than the corresponding path length of the driving element from the spoke to the end effector. A problem associated with this difference in path lengths is illustrated in FIG. 3. In FIG. 3, a portion the instrument 200 is illustrated in a rotated pose. In this rotated pose, the longitudinal axis 246 of the end effector is positioned at an angle a with respect to the longitudinal axis 242 of the shaft. In other words, the configuration of the end effector is angled with respect to the shaft. In this angled configuration, the driving elements 218, 220 remain taught around the joints of the instrument. However, the path length of the electrical cable is longer than the corresponding path length of the driving element and so the electrical cable may experience buckling in this pose. Buckling within the context of this application is defined as the deformation of a component (i.e., the electrical cable) under load. The deformation of the electrical cable may otherwise be referred to the bending or flexion of the cable outside of its defined path. The buckling of the electrical cable in FIG. 3 is illustrated by the deformation 248, which causes path of the electrical cable to jut out from the groove 236 in the electrical insulation within which the cable is meant to be housed.

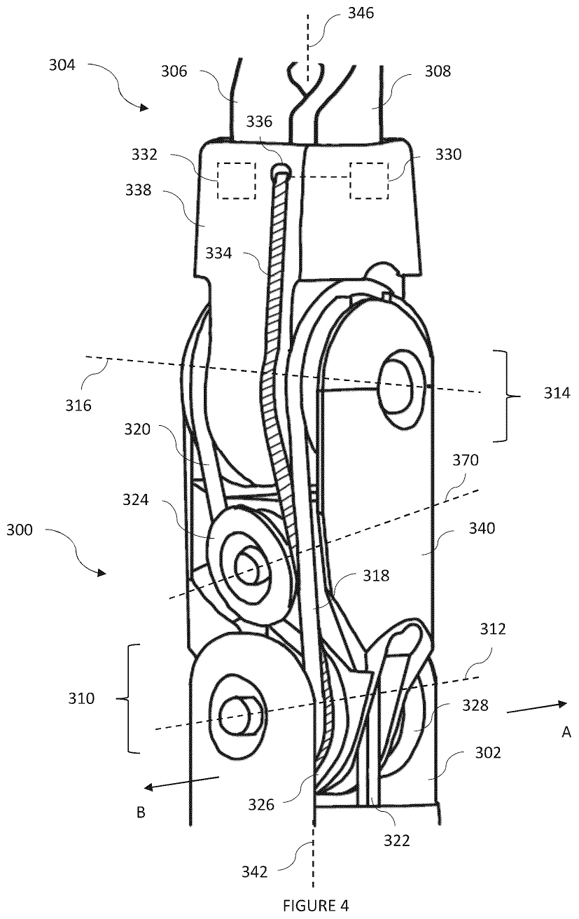

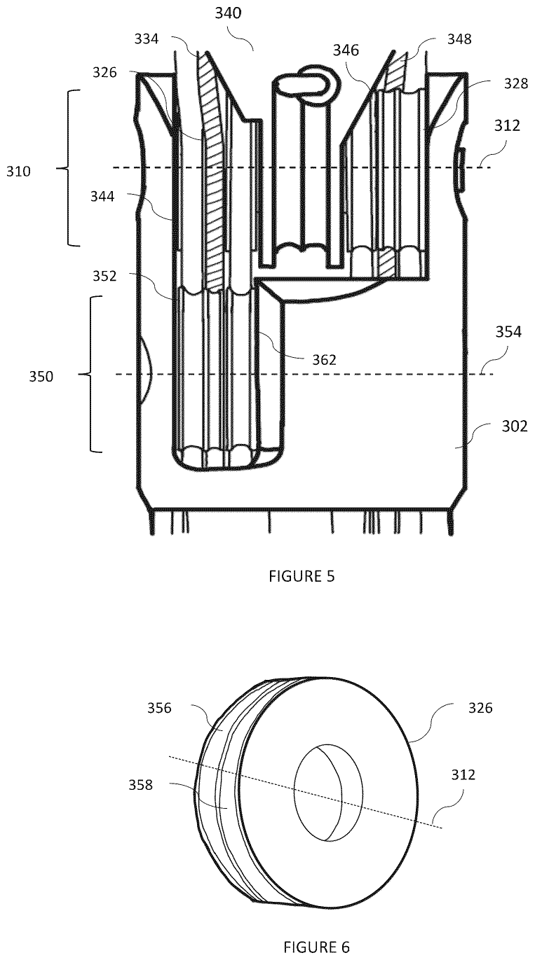

An additional problem with the configuration of the surgical instrument illustrated in FIG. 2 is that the electrical cable experiences friction as it passes through the distal end of the instrument. The distal end of the instrument in this context refers to the combination of the supporting body 240 and the end effector 204. In FIG. 3, at the distal end of the instrument, the electrical cable 234 passes through the supporting body 240 and out of an opening 244 in the supporting body towards the end effector 204. The electrical cables of the instrument 200 are formed of an inner core of electrically conductive material that is surrounded by an external casing of insulating material. When the instrument is in a rotated pose such as the one illustrated in FIG. 3 the electrical cable 234 may rub against the opening 244, leading to the wearing down of the insulating material of the cable. The friction between the electrical cable 234 and the opening 244 of the supporting body may cause the path of the cable through the instrument to deviate from its expected path, which also results in buckling. This friction also means that the cable as a whole may eventually fatigue to the point of failure. The abovementioned problems may be overcome by providing an arrangement for a robotic electrosurgical instrument 300 as illustrated in FIGS. 4 and 5. The electrosurgical instrument 300 in FIGS. 4 and 5 corresponds substantially to the one described with reference to FIG. 2 above. The electrosurgical instrument 300 is configured to be connected to a surgical robot. The electrosurgical instrument 300 is a robotic surgical instrument that comprises a shaft 302 and an end effector 304. In FIG. 3, the end effector 304 has a pair of end effector elements 306, 308 that are the same as the end effector elements 206, 208 illustrated in FIG. 2. It will be appreciated that, in other examples of the invention, the end effector 304 may comprise a single end effector element. In FIG. 3, the end effector elements 306, 308 may be first and second jaws of the end effector. The end effector 304 is connected to the distal end of the shaft 302 of the instrument by an articulation. The shaft 302 is connected at its proximal end to an interface for attaching to a robot arm. The articulation comprises joints 310, 314 that permit movement of the end effector 304 relative to the shaft 302. The articulation comprises a supporting body 340 which is the same as the supporting body 240 described with respect to FIG. 2. The instrument illustrated in FIG. 4 is in a straight configuration. In this configuration, the end effector 304 is aligned with the shaft 302 of the instrument. That is, the longitudinal axis 346 of the end effector is coincident with longitudinal axis 342 of the shaft.

The end effector 304 further comprises at least one electrical component. In one example, the at least one electrical component may be the metal jaws of the end effector elements 306 and 308. The electrical component has the same configuration and function as the electrical component described with respect to FIG. 2. In the example illustrated in FIG. 3, the instrument comprises a pair of electrical components. Thus, the instrument of FIG. 3 is a bipolar instrument. A first electrical component may be positioned on a first side of the end effector, and a second electrical component may be positioned on a second side of the end effector that opposes the first side of the end effector. Where an end effector comprises two end effector elements, a first electrical component may be (or may be attached to) the first end effector element and a second electrical component may be (or may be attached to) the second end effector element. The instrument comprises an external insulator 338 configured to insulate the electrically charged components of the instrument (e.g., the electrical components) from other parts of the surgical instrument and from the patient. The external insulator 338 may comprise a groove 336 within which the electrical cable is housed, which is the same as the groove 236 of the instrument 200 in FIG. 2.

The instrument 300 comprises first, second, third and fourth joints that are the same as the corresponding joints described with respect to the instrument 200 in FIG. 2. Each joint of the instrument is driven by at least one driving element. Each joint of the instrument may be driven by a pair of driving elements. Each joint of the instrument may be independently driven. The first joint 310 is driven by at least one driving element 322. The second joint 314 is driven by at least one driving element 318. The third joint is driven by at least one driving element 320. Thus, the first, second and third joints of the instrument are independently driven. The driving elements are elongate elements which extend from the joints in the articulation through the shaft to the instrument interface. Suitably, each driving element can be flexed laterally to its main extent at least in those regions where it engages the internal components of the articulation and instrument interface. In other words, each driving element can be flexed transverse to its longitudinal axis in those specific regions. This flexibility enables the driving elements to wrap around the internal structure of the instrument. The driving elements may be wholly flexible transverse to their longitudinal axes. The driving elements are not flexible along their main extents. The driving elements resist compression and tension forces applied along their length. In other words, the driving elements resist compression and tension forces acting in the direction of their longitudinal axes. Thus, the driving elements are able to transfer drive from the instrument interface to the joints. The driving elements may be cables.

A number of driving elements are wrapped around the first joint 310. For example, the first driving element 318 is wrapped around the first joint. The first driving element 318 is configured to drive a second joint 314 of the end effector about a second axis 316. The second joint 314 and second axis 316 are the same as the respective second joint 214 and second axis 316 referred to with respect to FIG. 2. The first driving element 318 is an elongate element that extends from the second joint 314, through the shaft 302 to the interface between the instrument and the robot arm. The first driving element 318 has a length that extends in its elongate direction and a cross-sectional area that is perpendicular to its length. The cross-sectional area of the first driving element 318 may be of any suitable shape. In one example, the cross-sectional area is circular in shape. The cross-sectional area of the first driving element 318 has a width. Where the cross-sectional area of the first driving element is circular, the width of the first driving element is the diameter of the first driving element. The first driving element 318 can be flexed laterally to its main extent at least in the regions where it engages the internal components of the articulation and instrument interface. This flexibility enables the driving element to wrap around the internal structure of the instrument.

The first driving element may be wholly flexible transverse to its longitudinal axis. The first driving element is not flexible along its main extent. The first driving element resists compression and tension forces applied along its length. In other words, the first driving element resists compression and tension forces acting in the direction of its longitudinal axes. Thus, the first driving element is able to transfer drive from the instrument interface to the joints. The first driving element may be a cable.

The elongate nature of the first driving element 318 is such that its passage through the instrument defines a path that extends at least partially around the first joint 310. In other words, the driving element may wrap at least partially around the first joint 310. Where the first joint 310 comprises one or more pulleys, the passage of the first driving element 318 may extend at least partially around a pulley 326 if the first joint. The pulley 326 is described in further detail below. The passage of the driving element through the instrument is defined by the longest dimension of the driving element. The passage of the first driving element 318 in FIG. 3 passes through the shaft 302, out of an opening in the distal end of the shaft, around the first joint 310, and then around the second joint 314.

As with the instrument in FIG. 2, the instrument 300 is supplied with electrical current by at least one electrical cable 334 that extends along (e.g., through) the shaft 302. The electrical cable 334 is attached at its first end to the instrument and at its second end to a source of electrical current (e.g., alternating current), that may be part of and located at the base of the surgical robot, for example. In an alternative example, the source of electrical current may be a standalone unit that is separate to the surgical robot. The electrosurgical instrument further comprises an electrical connector 330 that provides an electrical connection between the electrical cable and the electrical component of the end effector(s). The electrical connector 330 is the same as the electrical connector 230 described above with respect to FIG. 2.

As with the driving elements, the first electrical cable 334 is of an elongate nature. The first electrical cable 334 has a length that extends in its elongate direction and a cross-sectional area that is perpendicular to its length. The cross-sectional area of the first electrical cable 334 may be of any suitable shape. In one example, the cross-sectional area is circular in shape. The cross-sectional area of the first electrical cable 334 has a width. Where the cross-sectional area of the first electrical cable 334 is circular, the width of the first electrical cable is the diameter of the first electrical cable.

The passage of the first electrical cable 334 through the instrument defines a second path. The passage of the first electrical cable 334 through the instrument is defined by the longest dimension of the electrical cable. The passage of the first electrical cable 334 through the instrument 300 passes from the spoke of the driving element to which it the cable attached, through the body of the shaft 302, up through an opening in the distal end of the shaft (corresponding to opening 244 of the instrument 200 in FIG. 3) and towards the first joint 310. When it reaches the first joint 310, the passage of the electrical cable follows a path that is parallel to the first path (of the first driving element 318) around the first joint. The passage of the first electrical cable 334 follows a path that is parallel to the first path around at least a portion of the first joint 310. This can be seen both in FIG. 4 and in FIG. 5. Thus, the passage of the first electrical cable 334, when viewed from a plane that is perpendicular to the first axis 312, is coincident with the path of the first driving element (i.e., the first path) around the first joint 310. The path of the first electrical cable 334 is also offset from the path of the first driving element 318 along the first axis 312. That is, along the first axis 312, the path of the first electrical cable 334 is separated from the path of the first driving element 318 by a non-zero distance. The first electrical cable 334 is located further along the first axis 312 than the first driving element 318. The first electrical cable 334 may be located further along the first axis 312 in first direction A. The first electrical cable 334 may alternatively be located further along the first axis 312 when viewed from a second direction B.

By arranging the passage of the first electrical cable 334 so that it is parallel to the passage of the first driving element 318 around the first joint 310, the difference between the path length of the electrical cable 334 and that of the first driving element 318 can be reduced. By routing it around a joint of the instrument, the electrical cable can be pulled taught around that joint and excess slack in the cable can be reduced. Thus, when the instrument is articulated, there is less surplus length in the cable and thus the cable does is less likely to bulge out of the instrument when the driving elements are rotated about the pulley. Thus, buckling of the electrical cable is minimised and wearing of the cable can be reduced.

In some examples, the instrument may comprise a single electrical cable 234. The single electrical cable 234 may provide electrical energy to the end effector 304. In other examples, the instrument may comprise two electrical cables. In these examples, each electrical cable may supply electrical energy to a respective electrical component of the end effector. A second electrical cable is illustrated by reference 348 in FIG. 5. Where the instrument comprises a second electrical cable 348, it may also comprise a second electrical connector 332 for providing an electrical connection between the electrical cable and the electrical component of the end effector. The second electrical connector 332 performs the same function with respect to the second electrical cable 348 as the first electrical connector 330 performs for the first electrical cable 334. The elongate nature of the second electrical cable 348 is the same as that of the first electrical cable 334.

The first joint 310 comprises a length that extends along the first axis 312 and a cross-sectional area that is perpendicular to the first axis. The first joint 310 may be cylindrical shape. That is, the cross-sectional area of the first joint 310 may be circular in a plane that is perpendicular to the first axis. The cross-sectional area of the first joint 310 may be consistent across the length of the first joint. That is, the cross-sectional area of the first joint 310 may be the same at all points along the length of the first joint. Alternatively, the cross-sectional area of the first joint 310 may vary along the length of the joint. The first joint may comprise a plurality of components. The first joint 310 may comprise a cylindrical pin around which the first, second and third driving elements extend. The first path of the first driving element 318 and the second path of the first electrical cable 334 may each be least partially circumferential around the first joint. That is, the path of the first driving element 318 and the path of the first electrical cable 334 may extend at least partially around the circumference of the first joint 310. This means that the paths of both the first driving element 318 and the first electrical cable 334 have a partially circular path around the first joint 310.

The first joint 310 may additionally comprise a first set of pulleys. The first set of pulleys may comprise one or more pulleys. The first set of pulleys may comprise a first pulley 326 which is rotatable about the first axis 312. The first driving element 318 may be wrapped around the first pulley 326. In other words, the path of the first driving element 318 may wrap at least partially around the first pulley 326. The first pulley 326 may be cylindrical in shape. That is, the first pulley 326 may comprise a length that extends along the first axis 312 and a circular cross-sectional area in a plane that is perpendicular to the first axis. The cross-sectional area of the first pulley 326 may comprise a circular circumference.

An example of the first pulley 326 is illustrated in further detail in FIG. 6. The first pulley 326 may comprise a first groove 356. The first groove 356 may extend at least partially around the circumference of the first pulley 326. The first groove 356 may extend fully around the circumference of the first pulley 326. In other words, the first groove 356 may extend around the entirety of the circumference of the first pulley 326. The first groove 356 may be defined as a narrow depression that extends around the circumference of the first pulley. The first groove 356 may have a width that extends along the first axis 312, and a depth that extends radially towards the centre point of the circular cross-sectional area of the pulley. The depth of the first groove 356 defines a path of the first groove which has a circumference that is smaller than the circumference around the edge of the first pulley. The purpose of the first groove 356 may be to house the first driving element. That is, where the first driving element 318 wraps around the first pulley 326, the driving element may be at least partially housed within the first groove 356. This means that, where the driving element contacts the first pulley, it contacts the first groove 356 of the first pulley. The width of the first groove 356 may therefore be wider than the width (or diameter) of the first driving element 318. Alternatively, the depth of the first groove may be greater than the width (or diameter) of the first driving element. The first groove 356 ensures that the first driving element 318 is not displaced when it rotates around the first pulley 326. In other words, the first driving element 318 is pulled taught about the first pulley 326 when it rotates about the first pulley. In some examples, the first groove may extend entirely around the circumference of the first pulley. In other examples, the first groove may extend partially around the circumference of the first pulley. For example, the first groove may extend 180 degrees around the first pulley. The first groove may extend around the first pulley to any suitable degree. In a specific example, the first groove may extend 115 degrees around the circumference of the first pulley.

In one example, the first pulley 326 may further comprise a second groove 358. The second groove 358 may be offset from the first groove along the first axis 312. In other words, the first groove 356 may be located at a different distance along the first axis 312 to the second groove 358. As with the first groove, the second groove 358 may have a width that extends along the first axis 312, and a depth that extends radially towards the centre point of the circular cross-sectional area of the pulley. The depth of the second groove 358 defines a path of the second groove which has a circumference that is smaller than the circumference around the edge of the first pulley 326. Th second groove 358 may be said to extend around an outer circumference of the first pulley 326. The first pulley 326 may comprise different circumferences at different distances along its length. The first pulley 326 may comprise a first, maximum, circumference and two smaller circumferences. The two smaller circumferences may be located within the first and second grooves and may be defined by the depths of the first and second grooves. Thus, the first and second grooves may each define an outer circumference of the first pulley 326. The second groove 358 may be separated from the first groove 356 along the first axis 312 by a non-zero distance. At the same time, the first groove 356 may be axially aligned with the second groove 358 along the first axis. That is, the centre of the circle defining the cross-sectional area of the first groove 356 may be coincident with the centre of the circle defining the cross-sectional area of the second groove 358. In other words, the centre point of the first groove 356 may be located on the same axis as the centre point of the second groove 358. The first and second grooves may therefore extend around the same axis. More specifically, the first and second grooves may both extend around the first axis 312. As with the first groove, second groove may extend entirely or partially around the circumference of the first pulley.

The purpose of the second groove 358 may be to house the first electrical cable 334. That is, where the first electrical cable 334 wraps around the first pulley 326, the electrical cable may be at least partially housed within the second groove 358. This means that, where the first electrical cable 334 contacts the first pulley 326, it contacts the second groove 358 of the first pulley. The second groove 358 ensures that the first electrical cable 334 is not displaced when it rotates around the first pulley. In other words, the first electrical cable 334 is pulled taught about the first pulley 326 when it rotates about the first pulley.

Providing a pulley with first and second grooves for the driving element and electrical cable respectively is advantageous as it provides a simple and reliable mechanism for ensuring that the paths of the driving element and electrical cable are aligned around the first joint.

As mentioned above, the path of the electrical cable 334 through the instrument 300 extends parallel to the path of the first driving element 318 around the first joint 310. In some examples, the electrical cable 334 has the same width as the first driving element 318. Where the electrical cable has the same width as the first driving element, the path of the first groove 356 around the outer circumference of the first pulley 326 may have a radius that is the same as the radius of the path of the second groove 358 around the outer circumference of the first pulley. In other words, the depth of the first groove 356 may be the same as the depth of the second groove 358 around the first pulley 326. By ensuring that the first and second grooves have the same depth, it can be ensured that the paths of the first driving element 318 and the electrical cable 334 are parallel to each other around the first joint 310 in the example where both of these components have the same width.

In other examples, the electrical cable 334 may have a different width to that of the first driving element 318. The electrical cable 334 may have a smaller width than the first driving element 318. Where the electrical cable 334 has a smaller width than the first driving element 318, the path of the first groove 356 around its outer circumference of the first pulley 326 may have a radius that is smaller than the radius of the path of the second groove 358 around its outer circumference of the first pulley. In other words, the depth of the first groove 356 may be greater than the depth of the second groove 358. The increased depth of the first groove 356 relative to the second groove 357 accounts for the greater width of the driving element 318 with respect to the electrical cable 334. It ensures that the path of the electrical cable 334 through the instrument 300 extends parallel to the path of the first driving element 318 around the first joint in spite of the difference in the widths of these two components.

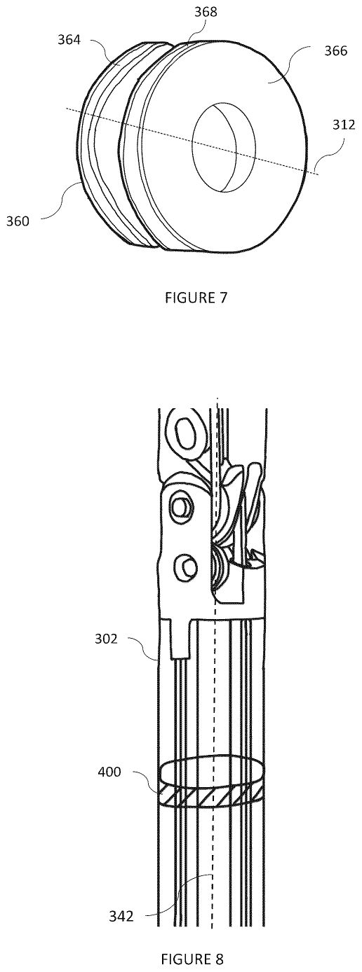

The first pulley may, instead of having first and second grooves, comprise only a single groove. The single groove of the first pulley may correspond to the first groove 356 described above with respect to FIG. 6 and may be configured to house the first driving element 318. The first joint 310 may further comprise a second pulley. More specifically, the first set of pulleys may comprise a second pulley in addition to the first pulley. An example of a two-pulley configuration for the first joint is illustrated in FIG. 7.

The first pulley 360 of the two-pulley configuration in FIG. 7 may be substantially the same as the first pulley 326 in FIG. 6. The first pulley 360 differs from the corresponding pulley 326 in that it comprises a single groove 364 for the first driving element. The first pulley 360 does not comprises a second groove for the electrical cable. The two-pulley configuration further comprises a second pulley 366 comprises a third groove 368. The second pulley 260 may be is rotatable about the first axis 312 independently of the first pulley 366. Thus, the first and second pulleys are separate components that can rotate separately to each other about the first axis.

The second pulley 366 corresponds broadly to the first pulley 360. The second pulley 366 may be cylindrical in shape. That is, the second pulley 366 may comprise a length that extends along the first axis 312 and a circular cross-sectional area in a plane that is perpendicular to the first axis 312. The third groove 368 may be offset from the first groove 364 along the first axis 312. In other words, the first groove 364 may be located at a different distance along the first axis 312 to the third groove 368. As with the first groove, the third groove 368 may have a width that extends along the first axis 312, and a depth that extends radially towards the centre point of the circular cross-sectional area of the second pulley 366. The depth of the third groove 368 defines a path of the third groove which is a circumference that is smaller than the circumference around the edge of the second pulley. The third groove 368 is configured to house the electrical cable. The third groove 368 may be said to extend around an outer circumference of the second pulley. The third groove 368 may be separated from the first groove along the first axis 312 by a non-zero distance. The third groove 368 is separated from the first groove by virtue of the first and third grooves being located on different pulleys. At the same time, the first groove 364 may be axially aligned with the third groove 368 along the first axis 312. That is, the centre of the circle defining the cross-sectional area of the first groove 364 may be coincident with the centre of the circle defining the cross-sectional area of the third groove 368. In other words, the centre point of the first groove may be located on the same axis as the centre point of the third groove 368. The first and third grooves may therefore extend around the same axis. More specifically, the first and third grooves may both extend around the first axis 312. As with the first groove, the third groove may extend entirely or partially around the circumference of the second pulley.

It has been mentioned above that, in some examples, the electrical cable 334 has the same width as the first driving element 318. Where the electrical cable has the same width as the first driving element, the path of the first groove 364 around the outer circumference of the first pulley 360 may have a radius that is the same as the radius of the path of the third groove 368 around the outer circumference of the second pulley. In other words, the depth of the first groove 364 around the first pulley 360 may be the same as the depth of the third groove 368 around the second pulley 366. By ensuring that the first and third grooves have the same depth, it can be ensured that the paths of the first driving element 318 and the electrical cable 334 are parallel to each other around the first joint 310 in the example where both of these components have the same width.

In other examples, the electrical cable 334 may have a different width to that of the first driving element 318. The electrical cable 334 may have a smaller width than the first driving element 318. Where the electrical cable 334 has a smaller width than the first driving element 318, the path of the first groove 364 around its outer circumference of the first pulley 360 may have a radius that is smaller than the radius of the path of the second groove 368 around its outer circumference of the second pulley 366. In other words, the depth of the first groove 364 may be greater than the depth of the third groove 368. The increased depth of the first groove 364 relative to the third groove 368 accounts for the greater width of the driving element 318 with respect to the electrical cable 334. It ensures that the path of the electrical cable 334 through the instrument 300 extends parallel to the path of the first driving element 318 around the first joint in spite of the difference in the widths of these two components.

In some examples, the end effector 304 of the instrument 300 may comprise a single end effector element. In such examples, the end effector element may be a needle for suturing, a knife, a stapler or a cauteriser, or any other suitable surgical instrument. In this example, the instrument is a monopolar instrument. The instrument therefore requires a single electrical cable 334 to provide electrical current to its end effector. The passage of the electrical cable 334 through the instrument, as described above, defines a path that is offset from the first path along the first axis 312 and that extends parallel to the first path (of the first driving element 318) around the first joint 310. In other examples, the end effector may comprise opposing first and second end effector elements 306, 308. In such examples, the opposing end effector elements may be smooth jaws, serrated jaws, a gripper, a pair of sheers or any other suitable pair of elements. In these examples, the first end effector element may be rotatable about the second joint 314 and the second end effector element may be independently rotatable relative to the shaft about a third axis by means of a third joint. The third joint and the third axis may be the same as the corresponding third joint and third axis described with respect to the instrument 200 in FIG. 2.

In the example illustrated in FIG. 3, the instrument is a bipolar instrument. The instrument therefore comprises a first electrical component comprised within its first end effector element 306, and a second electrical component comprised within its second end effector element 308. In addition to the first electrical cable 334, the instrument 300 further comprises a second electrical cable 348 that is configured to provide electrical current to a second electrical component of the second end effector element 308. The second electrical cable 348 is configured to provide electrical current to the second electrical component in the same way that the first electrical cable 334 is configured to provide electrical current to the first electrical component. As mentioned above, the instrument 300 further comprises a second electrical connector 332 that performs the same function with respect to the second electrical cable as the first electrical connector performs with respect to the first electrical cable.

The surgical instrument may further comprise a second driving element 320 configured to drive the third joint of the instrument. As with the first driving element 318, the length of the second driving element 320 may define a third path that extends at least partially around the first joint 310. The passage of the second driving element 320 through the instrument is defined by the longest dimension of the driving element. The passage of the second driving element 320 in FIG. 3 passes through the shaft 302, out of an opening in the distal end of the shaft, around the first joint 310, and then around the third joint. In other words, the second driving element 320 may wrap at least partially around the first joint 310. More specifically, the second driving element 320 may wrap around a further pulley 344 of the first joint.

Similarly to the first electrical cable, the passage of the second electrical cable 348 through the instrument defines a fourth path. The passage of the second electrical cable 348 through the instrument is defined by the longest dimension of the electrical cable. The passage of the second electrical cable 348 through the instrument 300 passes from the spoke of the driving element to which it the cable attached, through the body of the shaft 302, up through an opening in the distal end of the shaft (corresponding to opening 244 of the instrument 200 in FIG. 3) and towards the first joint 310. When it reaches the first joint 310, the passage of the second electrical cable 348 follows a path that is parallel to the third path (of the second driving element 320) around the first joint. The passage of the second electrical cable 348 follows a path that is parallel to the third path around at least a portion of the first joint 310. Thus, the passage of the second electrical cable 348, when viewed from a plane that is perpendicular to the first axis 312, is coincident with the path of the second driving element (i.e., the third path) around the first joint 310. The path of the second electrical cable 348 is also offset from the path of the second driving element 320 along the first axis 312. That is, along the first axis 312, the path of the second electrical cable 348 is separated from the path of the second driving element 320 by a non-zero distance. The second electrical cable 348 is located further along the first axis 312 than the second driving element 320. The second electrical cable 348 may be located further along the first axis 312 in first direction A. The second electrical cable 348 may alternatively be located further along the first axis 312 when viewed from a second direction B. The paths of the second driving element 320 and the second electrical cable 348 around the first joint may be defined by first and/or second pulleys corresponding to pulley 326 and/or pulleys 360, 366.

It has been mentioned above that each joint in the surgical instrument may be driven, instead of by a single driving element, by a pair of driving elements. Specifically, the second joint may be driven by a pair of driving elements comprising the first driving element 318 and a first further driving element (not illustrated). The first further driving element may be routed about the first joint around a second pulley 328 located on an opposing side of the first joint to the first pulley, relative to the first axis 312. Each of the first and first further driving elements may be configured to rotate the end effector in an opposing direction. In this way, the length of the first further driving element may define a fifth path that extends at least partially around the first joint. The first joint of the instrument may comprise additional pulleys around which further driving elements of the first and second pairs of driving elements may extend. The fourth joint may comprise corresponding additional pulleys.

Similarly, the third joint may be driven by a pair of driving elements comprising the second driving element 320 and a second further driving element (not illustrated). The second further driving element may be routed about the first joint around the second pulley 328 located on an opposing side of the first joint to the first pulley, relative to the first axis 312. Each of the second and second further driving elements may be configured to rotate the end effector in an opposing direction. In this way, the length of the second further driving element may define a sixth path that extends at least partially around the first joint. The first joint may be driven by a pair of driving elements comprising the third driving element 322 and a third further driving element (not illustrated). Each pair of driving elements may be constructed as a single piece. Alternatively, each pair of driving elements may be constructed as separate pieces.

The instrument 300 may further comprise a third pulley 324. The third pulley 324 may be positioned between the first and second joints 310, 314 and may be angularly offset from the first and second joints. The third pulley 324 may be referred to as a redirecting pulley. The third pulley 324 may positioned so as to direct a driving element from the first joint 310 to the second/third joints 314. The third pulley 324 may rotate about a first redirecting pulley axis 370. The first redirecting pulley axis 370 is at an angle 0 to the first axis 312. The angle is such that the driving element that rotates around third pulley 324 is redirected from a take-off point of a pulley of the first joint 310 to a pick-up point on the second/third joint 314. A take-off point is the point at which a driving element loses contact with a pulley. A pick-up point is the point at which a driving element first contacts a pulley. In FIG. 4, the third pulley 324 is illustrated as directing a second driving element 320 of the instrument from the first pulley 326 of the first joint 310 to the third joint which shares an axis 316 with the second joint 314. In an alternative example, the third pulley 324 may direct the first driving element 318 from the first pulley 326 to the second joint 314.

Suitably, the third pulley 324 comprises a groove which seats the relevant driving element. The third pulley 324 may cause the driving element to wrap more fully around the second/third joint than would happen if the third pulley 324 was not there, thereby increasing the length of engagement between the driving element and the second/third joint. Thus, the relevant driving element has a greater travel around the second/third joint and is hence able to cause a larger rotation of the end effector element 304 about the second/third axis 316 than would be possible without the pulley 324. The third pulley 324 causes the pick-up point of the second driving element 320 on the second joint 314 to change relative to where it would have been without the redirecting pulley 324.

The passage of the electrical cable 334 through the instrument may be such that the electrical cable extends at least partially around the third pulley 324. In other words, the second path defined by the passage of the electrical cable through the instrument may extend at least partially around the third pulley 324. The groove comprised within the third pulley 324 that seats the second driving element may be further configured to seat the electrical cable. That is, in addition to the first driving element 318, the first electrical cable may extend at least partially around the third pulley 324. The first driving element and the electrical cable may extend around opposing sides of the third pulley. The first driving element may be diametrically opposed to the electrical cable. The first driving element and the electrical cable may extend symmetrically to each other about the first axis 310. In extending at least partially around the third pulley, the path of the electrical cable is further constrained in line with that of the driving elements of the instrument. Thus, the advantages through the reduced difference in path length between the electrical cable and the first driving element are further realised.

The surgical instrument may further comprise a fourth joint 350 corresponding to the fourth joint 224 in FIG. 2. The fourth joint 350 may comprise at least one pulley 352. Preferably, where the first joint comprises first pulley 326 and the further pulley 344, the fourth joint comprises a corresponding pair of pulleys 352, 362. The fourth joint 350 is located relative to the first joint 310 so as to ensure that the components that drive the first joint are retained in contact with the first joint 310. The pulleys of the fourth joint 350 may rotate about a fourth axis 354. The fourth axis 354 may be parallel to the first axis 312. The fourth axis is offset from the first axis 312 along the longitudinal axis 342 of the shaft.

FIG. 8 illustrates a cutaway of a portion of the instrument of FIGS. 4 and 5 that is proximal to the end effector of the instrument. That is, the distance between the portion of the instrument illustrated in FIG. 8 and the robot arm is less than the distance between the end effector 304 and the robot arm. From FIG. 8, it can be seen that the shaft 302 of the instrument may comprise a resilient barrier 400. More specifically, the resilient barrier 400 is located inside the shaft. The resilient barrier may extend across a cross-sectional area of the shaft. The cross-sectional area may be transverse to the longitudinal axis 342 of the shaft. Suitably, the outer edge of the resilient barrier 400 is in sealed contact with the interior wall of the shaft at all points around the outer edge of the cross-sectional area. Suitably, the resilient barrier 400 is fabricated from a material which inhibits the passage of fluid through it. Thus, the resilient barrier 400 acts as a stopper or a bung, inhibiting the passage of fluid (such as the insufflation gas) through the shaft from the side of the resilient barrier facing the articulation to the opposing side of the resilient barrier facing the interior of the shaft. The resilient barrier 400 is internal to the shaft 302. The resilient barrier 400 may be located anywhere along the shaft. The resilient barrier 400 may preferably be located in the end of the shaft 302 that is proximal to the end effector 304. The closer the resilient barrier 400 to the distal end of the shaft 302, the less insufflation gas that can leak from the surgical site into the instrument before being inhibited by the resilient barrier.

Each of the driving elements of the instrument 300 may pass through the resilient barrier 400. Where each joint is driven by a single driving element, each driving element passes through a hole in the resilient barrier. Suitably, each driving element passes through a channel that extends through the resilient barrier. The channel may be a cylindrical channel. Each driving element may be described as passing through a hole in the resilient barrier 400. In this example, the first driving element that drives the second joint is configured to pass through a first channel (or hole) that extends through the resilient barrier. Similarly, the second driving element that drives the third joint is configured to pass through a second channel (or hole) that extends through the resilient barrier. The electrical cable is configured to pass through a third channel (or hole) that extends through the resilient barrier. For the purposes of this application, the terms “channel” and “hole” will be considered to be synonymous. Where each joint is driven by a pair of driving elements, each driving element of each pair of driving elements passes through the resilient barrier.

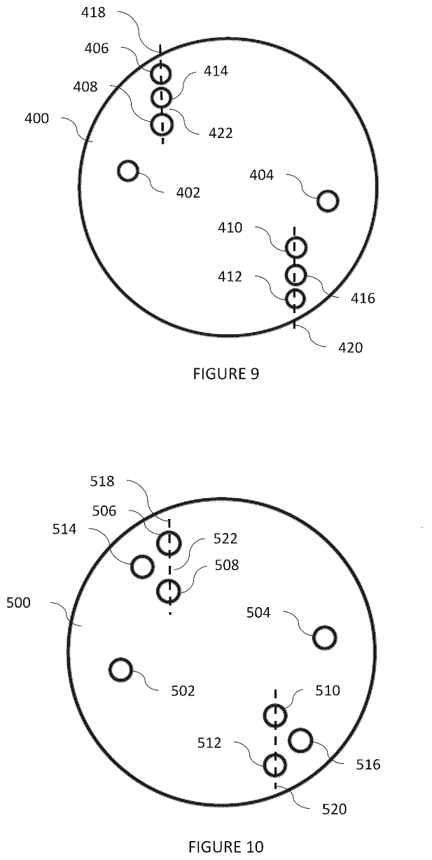

FIG. 9 illustrates an exemplary plan view of a resilient barrier 400. The plan view may illustrate a distal surface of the resilient barrier. The instrument comprising the resilient barrier of FIG. 4 comprises first pair of driving elements that drives its second joint, a second pair of driving elements that drives its third joint and a third pair of driving elements that drives its first joint. A first driving element of the third pair of driving elements passes through hole 402 of the resilient barrier, and a second driving element of the third pair of driving elements passes through hole 404 of the resilient barrier. A first driving element of the first pair of driving elements passes through hole 406, and a first driving element of the second pair of driving elements passes through hole 408. A second driving element of the first pair of driving elements passes through hole 410, and a second driving element of the second pair of driving elements passes through hole 412. Similarly, each electrical cable of the instrument 300 may pass through a hole in the resilient barrier. More specifically, a first electrical cable of the instrument may pass through hole 414, and a second electrical cable may pass through hole 416. The hole 414 through which the first electrical cable passes may be parallel to the holes 406, 408 through which the first and second pairs of driving elements pass. That is, the centre points of the holes 406, 408, 414 may intersect a common axis 418. In other words, the holes 406, 408, 414 may be aligned along a common axis. Similarly, the holes 410, 412, 416 may be aligned along a common axis. Hole 406 may otherwise be referred to as a first channel, hole 408 may otherwise be referred to as a second channel and hole 414 may otherwise be referred to as a third channel. Similarly, hole 410 may otherwise be referred to as a fourth channel, hole 412 may otherwise be referred to as a fifth channel and hole 416 may otherwise be referred to as a sixth channel.

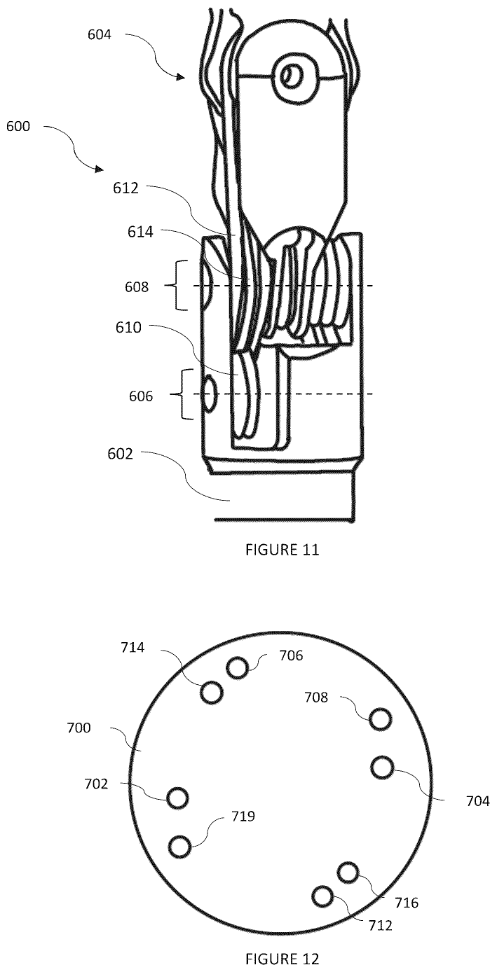

The general structure of a resilient barrier as illustrated in FIGS. 8 and 9 (as well as FIGS. 10 and 11) is that each driving element and electrical cable may pass through the resilient barrier without contacting the resilient barrier. This may be achieved, for example, by the diameter of each hole being greater than the diameter of the driving element which passes through it. In this case, the resilient barrier does not constrain the driving elements at all. In other words, there is minimal interaction between the resilient barrier and the driving elements. As a result of the holes not contacting the driving elements, the resilient barrier does not wholly seal the interior of the shaft from fluid contacting the resilient barrier from the surgical site. However, the resilient barrier provides an effective block to fluid so as to substantially block the passage of insufflation gas and hence reduce leakage rates. Alternatively, each driving element may pass through the resilient barrier contacting the resilient barrier. This may be achieved, for example, by the diameter of each hole being less than the diameter of the driving element which passes through it.

A problem with the arrangement of channels, or holes, in the resilient barrier 400 of FIG. 9 is that the parallel arrangement of holes 406, 408 and 414 means that they are located very close together. It is difficult to manufacture the resilient barrier with holes that are located this close together. The close proximity of the holes to each other means that, during manufacture, the resilient barrier could be prone to breakages in the spaces between proximal holes. An example of a space between proximal holes is space 422 between holes 406 and 408. Alternatively, it may be impossible to manufacture spaces between the holes such as the space 422.

The abovementioned problem may be avoided by manufacturing a resilient barrier 500 as illustrated in FIG. 10. The resilient barrier 500 is illustrated in FIG. 10 out of use, without any cables or driving elements passing through it. Similarly to resilient barrier 400, the resilient barrier 500 comprises holes, or channels, through which the driving elements and electrical cables of the instrument extend. A first driving element of the third pair of driving elements passes through hole (or channel) 502, and a second driving element of the third pair of driving elements passes through hole (or channel) 504. A first driving element of the first pair of driving elements passes through hole 506 (or channel), and a first driving element of the second pair of driving elements passes through hole (or channel) 508. A second driving element of the first pair of driving elements passes through hole (or channel) 510, and a second driving element of the second pair of driving elements passes through hole (or channel) 512. Similarly, each electrical cable of the instrument 300 may pass through a hole in the resilient barrier. More specifically, a first electrical cable of the instrument may pass through hole (or channel) 514, and a second electrical cable may pass through hole (or channel) 516.

In the resilient barrier 500, the channels 506, 508 through which the first pair of driving elements pass may be offset from the channel 514 through which the first electrical cable passes. Similarly, the channels 510, 512 through which the second pair of driving elements pass may be offset from the channel 516 through which the second electrical cable passes. All of the channels of the resilient barrier 500 may be cylindrical in shape. In other words, the channels may have a length that extends through the resilient barrier and a circular cross-sectional area. Alternatively, the channels may have an elliptical cross-sectional area. The circular/elliptical cross-sectional area may extend along a distal surface of the resilient barrier. The first channel 506 and the second channel 508 may be aligned similarly to the first and second channels 406, 408 in resilient barrier 400. That is, the centre points of the first and second channels may pass through a common axis, or line, 518. An advantage of the channels having an elliptical cross-sectional area is that the channels may be aligned across their width (i.e., with their shorter dimension extending along the common axis 518, and their longer dimension extending perpendicular to the common axis 518) to increase the area of material between the channels and thereby improve ease of manufacturing if the resilient barrier. The location of the first and second channels on the resilient barrier may correspond to the position of the corresponding driving elements when they pass through the barrier. The resilient barrier 500 differs from corresponding resilient barrier 400 in that the third channel 514 is offset from the common axis 518 through which (a) the centre point of the first channel extends, and (b) a centre point of the second channel extends. In other words, the third channel 514 is misaligned from the common axis 518 along which the centre points of the first and second channels extend.

Similarly to the first and second channels, the fourth channel 510 and the fifth channel 512 may be aligned in the same ways as the fourth and fifth channels 410, 412 in resilient barrier 400. That is, the centre points of the fourth and fifth channels may pass through a common axis, or line, 520. The sixth channel 516 is offset from the common axis 520 through which (a) the centre point of the fourth channel extends, and (b) a centre point of the fifth channel extends. In other words, the sixth channel 514 is misaligned from the common axis 520 along which the centre points of the fourth and fifth channels extend.

As the resilient barrier 500 in FIG. 10 is illustrated not in use, the resilient barrier can be manufactured as illustrated. An advantage of manufacturing the resilient barrier in this way is that holes, or channels, for the driving elements and electrical cables can be engineered whilst not subjecting the areas of the resilient barriers between the holes to breaking. That is, the spaces between the holes, or channel, in the resilient barriers is increased (see, for example, space 522), making manufacturing of the resilient barrier feasible.

An alternative solution for manufacturing the resilient barrier is illustrated in FIGS. 11 and 12. In FIG. 11, a configuration of the surgical instrument 600 is shown comprising a shaft 602 connected to an end effector 604 via an articulation. The surgical instrument 600 corresponds substantially to that in FIGS. 4 and 5 except for with respect to the difference described below. As described with respect to instruments 200 and 300 above, the instrument 600 comprises a fourth joint 606. The fourth axis 606 may be parallel to the first axis 608 and configured to perform the same function as the fourth joint described with respect to instruments 200 and 300.

The instrument 600 comprises a first pulley configured to house the first driving element (of the first pair of driving elements) 612, corresponding to first pulley 326 of the instrument 300. The instrument 600 further comprises a further pulley configured to house a second driving element (or the first driving element of the second pair of driving elements) 612, corresponding to the further pulley 344 of the instrument 300. The fourth joint comprises a first pulley 610 corresponding to the first pulley 372 of the first joint in instrument 300. However, the fourth joint does not comprise a further pulley that is configured to house the second driving element. In other words, the fourth joint comprises no further pulley configured to house the second driving element. Where the joints in the instrument are driven by pairs of driving elements, the fourth joint may also comprise a pulley for housing a further driving element of the first set of driving elements, but not a further pulley for housing a further driving element of the second set of driving elements. That is, the fourth joint only comprises pulleys for the first set of driving elements, and not for the second set of driving elements.

An advantage of configuring the instrument as illustrated in FIG. 11 is that the path of the second driving element(s) at the point where it passes through the resilient barrier is offset from its location when the fourth joint comprises its further pulley(s). This is illustrated in FIG. 12. In FIG. 12, channels 702 and 704 correspond to channels 402 and 404 in FIG. 9, respectively. Similarly, channels 706, 712, 714 and 716 correspond to channels 406, 412, 414 and 416, respectively. The channels may be circular or elliptical in cross-sectional area. The locations of channels 702-706 and 712-716 are substantially the same as the locations of corresponding channels 402-406 and 412-416. The arrangement of the resilient barrier in FIG. 12 differs from that of the one in FIG. 9 in that the channels 708 and 710 are offset from their locations in the barrier 400. These offsets are enabled by the displacement of the driving elements of the second pair driving elements due to the lack of further pulleys around the fourth joint 606. This offset spaces the channels 708 and 710 away from the channels 706, 714 and channels 712, 716 respectively and allows for the resilient barrier 700 to be manufactured without breaking.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description, it will be evident to a person skilled in the art that various modifications may be made within the scope of the invention.

1. A robotic electrosurgical instrument comprising:
a shaft;
an end effector comprising an electrical component, wherein the end effector comprises opposing first and second end effector elements;
a first joint permitting the end effector to rotate relative to the shaft about a first axis, the shaft terminating at the first joint;
a first driving element configured to drive the end effector about a second joint, the passage of the first driving element through the instrument defining a first path that extends at least partially around the first joint; and
an electrical cable configured to provide electrical current to the electrical component, the passage of the electrical cable through the instrument defining a second path that is offset from the first path along the first axis and that extends parallel to the first path around the first joint;
the first joint further comprising a first pulley that is rotatable about the first axis;
the first pulley comprising a first groove, wherein the first driving element is at least partially housed within the first groove; and
a second groove that is offset from and axially aligned with the first groove along the first axis, wherein the electrical cable is at least partially housed within the second groove.

2. The instrument as claimed in claim 1, wherein the first and second paths are at least partially circumferential around the first joint.

3. The instrument as claimed in claim 1, wherein the first pulley has a circular cross-sectional area in a plane that is perpendicular to the first axis.

4. The instrument as claimed in claim 1, wherein each of the first and second grooves extends around an outer circumference of the first pulley.

5. The instrument as claimed in claim 4, wherein the path of the first groove around the outer circumference of the first pulley has a radius that is the same as the radius of the path of the second groove around the outer circumference of the first pulley.

6. The instrument as claimed in claim 4, wherein the path of the first groove around the outer circumference of the first pulley has a radius that is smaller than the radius of the path of the second groove around the outer circumference of the first pulley.

7. The instrument as claimed in claim 1, wherein the first joint further comprises a second pulley, the second pulley comprising a third groove that is offset from and axially aligned with the first groove along the first axis, and wherein the electrical cable is at least partially housed within the third groove.

8. The instrument as claimed in claim 7, wherein the second pulley is rotatable about the first axis independently of the first pulley.

9. The instrument as claimed in claim 7, wherein the second pulley has a circular cross-sectional area in a plane that is perpendicular to the first axis.

10. The instrument as claimed in claim 7, wherein the third groove extends around an outer circumference of the second pulley.

11. The instrument as claimed in claim 10, wherein the path of the first groove around the outer circumference of the first pulley has a radius that is the same as the radius of the path of the third groove around the outer circumference of the second pulley.

12. The instrument as claimed in claim 10, wherein the path of the first groove around the outer circumference of the first pulley has a radius that is smaller than the radius of the path of the third groove around the outer circumference of the second pulley.

13. The instrument as claimed in claim 1, further comprising a third pulley that is positioned between the first and second joints and is angularly offset from the first and second joints, wherein the second path extends at least partially around the third pulley.

14. (canceled)

15. The instrument as claimed in claim 1, wherein the first end effector element is rotatable about the second joint and the second end effector element is independently rotatable relative to the shaft about a third axis by means of a third joint.

16. The instrument as claimed in claim 15, wherein the electrical component is a first electrical component comprised within the first end effector element and the electrical cable is a first electrical cable being configured to provide electrical current to the first electrical component, and wherein the surgical instrument further comprises:
a second driving element configured to drive the third joint, the length of the second driving element defining a third path that extends at least partially around the first joint;
a second electrical component comprised within the second end effector element; and
a second electrical cable configured to provide electrical current to the second electrical component;
wherein the passage of the second electrical cable through the instrument defines a fourth path that is offset from the third path along the first axis and extends parallel to the third path around the first joint.

17. (canceled)

18. The instrument as claimed in claim 1, wherein the first joint is driven by a pair of driving elements comprising the first driving element and a first further driving element, and wherein the length of the further driving element defines a fifth path that extends at least partially around the first joint.

19. The instrument as claimed claim 18, further comprising a resilient barrier inside the shaft extending over a cross-sectional area of the shaft, wherein each of the first and second driving elements is configured to pass through respective first and second channels that extend through the resilient barrier, and wherein the electrical cable is configured to pass through a third channel that extends through the resilient barrier.

20. The instrument as claimed in claim 19, wherein the first, second and third channels are cylindrical in shape, and wherein a centre point of the third channel is offset from a line that passes through (a) a centre point of the first channel and (b) a centre point of the second channel, along a distal surface of the resilient barrier.

21. The instrument as claimed in claim 13, wherein:
the first joint comprises the first pulley and a further pulley that is also rotatable about the first axis, the further pulley comprising a further groove configured to at least partially house the second driving element; and
the instrument further comprises a fourth joint located parallel to the first axis and offset from the first axis along the longitudinal axis of the shaft, wherein the fourth joint comprises a first pulley configured to house the first driving element and no further pulley configured to house the second driving element.

22. The instrument as claimed in claim 1, wherein the instrument is configured to be connected to a surgical robot.
